(19) United States
(12) Patent Application Publication
(10) Pub. No.: US 2006/0036237 A1
(43) Pub. Date: Feb. 16, 2006

(54) Devices and methods for selective orientation of electrosurgical devices

(75) Inventors: Terry S. Davison, Redwood City, CA (US); Matthew L. Yates, Mountain View, CA (US); Theodore C. Ormsby, Escondido, CA (US); Jean Woloszko, Mountain View, CA (US)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/144,934

(22) Filed: Jun. 3, 2005

Related U.S. Application Data
(63) Continuation of application No. PCT/US03/38782, filed on Dec. 3, 2003.
(60) Provisional application No. 60/430,946, filed on Dec. 3, 2002.

(51) Int. Cl. A61B 18/14

(52) U.S. Cl. 606/41; 606/49; 606/50

(57) ABSTRACT

Devices and methods for the selective orientation of electrodes for effecting the controlled ablation, coagulation, or other modification of a target tissue in vivo with no or minimal collateral tissue damage. The subject devices are electrosurgical wands configured to only bend in a single plane. The subject methods involve use of the subject devices to prepare for the treatment of a target tissue site.

The present application is a continuation of PCT/US2003/38782 filed Dec. 3, 2003 and claims priority to U.S. provisional application No. 60/430,946 filed Dec. 3, 2002. The present invention relates generally to the field of electrosurgery and, more particularly, to surgical devices and methods which employ high frequency voltage to cut, ablate, treat, or modify body tissue structures.

### FIELD OF THE INVENTION

### BACKGROUND OF THE INVENTION

Conventional electrosurgical methods are widely used since they generally reduce patient bleeding associated with tissue cutting operations and improve a surgeon's visibility. Traditional electrosurgical techniques for treatment have typically relied on thermal methods to rapidly heat and vaporize liquid within tissue and to cause cellular destruction. In conventional monopolar electrosurgery, for example, electric current is directed along a defined path from an exposed or active electrode through the patient's body to the return electrode that is attached externally to a suitable location on the patient's skin. Since the defined path through the patient's body has a relatively high electrical impedance, large voltage differences must typically be applied between the active and return electrodes to generate a current suitable for cutting or coagulation of the target tissue or fluid. This current, however, may inadvertently flow along localized pathways in the body having less impedance than the defined electrical path. This situation can result in damage to or destruction of tissue along and surrounding this pathway.

Bipolar electrosurgical devices have an advantage over monopolar devices because the return current path does not flow through the patient beyond the immediate site of application of the bipolar electrodes. In bipolar devices, both the active and return electrode are typically exposed so that they may both contact tissue, thereby providing a return current path from the active to the return electrode through the tissue. One drawback with this configuration, however, is that the return electrode may cause tissue desiccation or destruction at its contact point with the patient's tissue.

Another limitation of conventional bipolar and monopolar electrosurgery devices is that they are not suitable for the precise removal (i.e., ablation) of tissue. For example, conventional electrosurgical cutting devices typically operate by creating a voltage difference between the active electrode and the target tissue, causing an electrical arc to form across the physical gap between the electrode and tissue. At the point of contact between the electric arcs and the tissue, rapid tissue heating occurs due to high current density between the electrode and tissue. This high current density causes cellular fluids to rapidly vaporize into steam, thereby producing a “cutting effect” along the pathway of localized tissue heating. The tissue is parted along the pathway of evaporated cellular fluid, inducing undesirable collateral tissue damage in regions surrounding the target tissue site.

The use of electrosurgical procedures (both monopolar and bipolar) in electrically conductive environments can be further problematic. For example, many procedures require flushing of the region to be treated with isotonic saline, both to maintain an isotonic environment and to keep the field of view clear. However, the presence of saline, which is a highly conductive electrolyte, can cause shorting of the active electrode(s) in conventional monopolar and bipolar electrosurgery. Such shorting causes unnecessary heating in the treatment environment and can further cause non-specific tissue destruction.

Conventional electrosurgical techniques used for tissue ablation also suffer from an inability to control the depth of necrosis in the tissue being treated. Most electrosurgical devices rely on creation of an electric arc between the treating electrode and the tissue being cut or ablated to cause the desired localized heating. Such arcs, however, often create very high temperatures causing a depth of necrosis greater than 500 μm, frequently greater than 800 μm, and sometimes as great as 1700 μm. The inability to control such depth of necrosis is a significant disadvantage in using electrosurgical techniques for tissue ablation.

To address the drawbacks of such convention electrosurgical devices and techniques, the assignee of the present invention, ArthroCare, Inc., has developed an advanced bipolar radiofrequency (RF) ablation technology. This technology, commercially known as Coblation® technology, is non-heat driven but, instead, causes molecular disintegration of the target tissue structure. The ablation process involves the application of RF energy between active and return electrodes (integrally configured within a wand-type device) via a conductive medium, usually saline, causing a plasma field or layer to form at the tissue surface. The saline may be delivered via a channel integrally arranged with the electrodes. An aspiration channel may also be integrally provided in the Coblation® device to remove excess saline as well as to remove tissue fragments from the operative site, sometimes by ablating the fragments with a digestion electrode. Rather than forming a conductive path through the tissue, the current passing between the active electrode and the return electrode travels via the conductive medium (e.g., the saline) to form an ionized gas or plasma field. As discussed herein, the plasma field causes molecular dissociation (rather than thermal evaporation or carbonization) of the target tissue structure. Thereby, tissue is volumetrically removed through molecular disintegration of larger organic molecules into smaller molecules and/or atoms, such as hydrogen, oxygen, oxides of carbon, hydrocarbons and nitrogen compounds. Because the current does not pass directly through tissue during the Coblation® process, tissue heating is minimal, remaining below 70° C., thereby minimizing collateral tissue damage as the result of undesired heating. Most of the current is consumed in the plasma layer by an ionization process. As such, the plasma field becomes saturated with highly ionized particles which have sufficient energy to break organic molecular bonds within tissue.

Coblation® technology is effective and advantageous in any surgical application where rapid healing, reduced post-operative pain and controlled and efficient ablation are desired. In particular, Coblation® has applications in general surgery, arthroscopy, cardiovascular applications, urology and ears, nose and throat (ENT), spinal surgery and dermatological procedures. Examples of such applications are described in U.S. Pat. Nos. 5,697,882; 5,843,019; 5,871,469; 6,142,992; 6,149,620; 6,224,592; 6,235,020; 6,416,508 all of which are incorporated by reference herein.

In certain surgical applications, the target ablation site may be somewhat difficult to reach and require specially designed and shaped instruments to effectively ablate the tissue. Certain conventional electrosurgical devices are provided with preformed angular configurations to better access the target site. Still other electrosurgical instruments employ bendable electrodes or malleable shafts which may be bent or oriented in any direction (i.e., three dimensional orientation). Such devices include standard Bovie devices and other conventional ablation devices that subscribed to the conventional ablation techniques discussed above. There remains a need to control the bending or orientation of such devices to a pre-determine plane or configuration. This need is even more evident with ablation devices, such as the Coblation® device described, or other multifunctional surgical instruments that provide multiple integral components.

The present invention teaches another approach to selectively orienting ablation devices. In one variation of the invention, devices having particularly configured tissue treatment surfaces or components, e.g., electrodes, and/or integral channels for the delivery and removal of material, such as the Coblation® device described above, may be selectively orientated according to the present invention. Ideally, any of these devices are manufacturable at a relatively low cost. The present invention provides such apparatus and methods, as is described in enabling detail herein below.

### SUMMARY OF THE INVENTION

The present invention includes devices and methods for the selective orientation of surgical instruments. Variations of the invention are useful in medical devices having a multiple-component configuration where such components are desirably maintained in a position, configuration or orientation relative to each other or where such components are highly subject to less optimal function if subject to an excessive bending force. A particular embodiment of the present invention is a device and method for the selective orientation of a shaft of a device in a single plane for effecting locating a tissue treatment surface of the device to provide a controlled ablation, coagulation, or other modification of a target tissue in vivo.

An apparatus according to the present invention generally includes an electrosurgical instrument having a shaft with proximal and distal end portions, a tissue treatment surface at a distal end portion, the tissue treatment surface having one or more active electrode(s) at the distal end portion; the device may further include one or more connectors coupling the active electrode(s) to a source of high frequency electrical energy. Alternatively, the device may have an integral cable coupling the active electrode(s) to a source of high frequency electrical energy. The instrument comprises probes or wands designed for direct use in either open procedures, percutaneous procedures, minimally invasive or arthroscopic access type procedures. The apparatus may further include a supply or source of an electrically conductive medium, including a fluid, gel, etc. The conductive medium may be an isotonic saline, blood, extracelluar or intracellular fluid, delivered to, or already present at, the target site. Alternatively, or in combination, a viscous medium, such as a gel, may be applied to the electrodes of the device prior to approaching the target site. The electrically conductive medium allows for a current flow path to form between the active electrode(s) and one or more return electrode(s). In one embodiment, the return electrode is spaced a sufficient distance from the active electrode(s) to substantially avoid or minimize current shorting therebetween, and to shield the tissue at the target site from the return electrode. The spacing of the return electrode may be such that it is spaced away and not in contact with the target tissue.

Other features, aspects and variations of the invention will become apparent to those skilled in the art upon reading this disclosure in combination with the accompanying figures.

### BRIEF DESCRIPTION OF THE FIGURES

To facilitate understanding, the same reference numerals have been used (where practical) to designate similar elements that are common to the Figures. Some such numbering has, however, been omitted for the sake of drawing clarity.

FIG. 1 illustrates an embodiment of an electrosurgical apparatus of the present invention.

FIG. 2 is a partial longitudinal cross-section view of the handle and shaft portions of the device of FIG. 1.

FIGS. 3A is a side views of the working distal end of the device of FIG. 1. FIG. 3B is an end view of the working distal end of FIG. 3A.

FIG. 4 is a perspective view of a bendable orientation reinforcing member of the present invention.

FIG. 5 is cross-sectional view of the shaft of the electrosurgical apparatus of FIG. 1 taken along the arrows A-A.

FIGS. 6A-6D illustrate additional variations of reinforcing members of the present invention.

FIGS. 7 illustrate an example of reinforcing members being bendable only about a distal portion thereof.

### DETAILED DESCRIPTION OF PREFERRED EMBODIMENTS

In further describing the subject invention, an overview of Coblation® technology is provided followed by a description of the subject devices and systems, the subject methods and a summary of the kits which include the subject devices for performing the subject methods.

Before the present invention is described in detail, it is to be understood that this invention is not limited to particular variations set forth herein as various changes or modifications may be made to the invention described and equivalents may be substituted without departing from the true spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise; It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

As noted above, prior to embarking on a description of specific embodiments of the present invention, an overview of Coblation® technology is provided. In procedures in which Coblation® technology is employed, a high frequency voltage difference is applied between one or more active electrode(s) and one or more return electrode(s) to develop high electric field intensities in the vicinity of the target tissue. The high electric field intensities may be generated by applying a high frequency voltage that is sufficient to vaporize an electrically conductive medium over at least a portion of the active electrode(s) in the region between the distal tip of the active electrode(s) and the target tissue. The electrically conductive medium may be, for example, a liquid, gel or gas. Such electrically conductive medium include isotonic saline, blood, extracelluar or intracellular fluid, delivered to, or already present at, the target site, or a viscous medium, such as a gel, applied to the target site.

When the conductive medium is heated enough such that atoms vaporize off the surface faster than they recondense, a gas is formed. When the gas is sufficiently heated such that the atoms collide with each other and knock their electrons off in the process, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the plasma or vapor layer becomes sufficiently low (i.e., less than approximately 1020 atoms/cm3 for aqueous solutions), the electron mean free path increases to enable subsequently injected electrons to cause impact ionization within the vapor layer). Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are hotter than the ions. Thus, the electrons, which are carried away from the tissue towards the return electrode, carry most of the plasma's heat with them, allowing the ions to break apart the tissue molecules in a substantially non-thermal manner.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of this phenomena can be found in commonly assigned U.S. Pat. No. 5,697,882 the complete disclosure of which is incorporated herein by reference.

In some applications of the Coblation technology, high frequency (RF) electrical energy is applied in an electrically conducting media environment to shrink or remove (i.e., resect, cut, or ablate) a tissue structure and to seal transected vessels within the region of the target tissue. Coblation technology is also useful for sealing larger arterial vessels, e.g., on the order of about 1 mm in diameter. In such applications, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue. In other applications, an electrosurgical instrument is provided having one or more coagulation electrode(s) configured for sealing a severed vessel, such as an arterial vessel, and one or more active electrodes configured for either contracting the collagen fibers within the tissue or removing (ablating) the tissue, e.g., by applying sufficient energy to the tissue to effect molecular dissociation. A single voltage can be applied to the tissue by the coagulation electrode(s), as well as to the active electrode(s) to ablate or shrink the tissue. In certain applications, the power supply is combined with the coagulation instrument such that the coagulation electrode is used when the power supply is in the coagulation mode (low voltage), and the active electrode(s) are used when the power supply is in the ablation mode (higher voltage).

The amount of energy produced by the Coblation® technology may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the medium in contact with the electrodes; density of the medium; and other factors. Accordingly, these factors can be manipulated to control the energy level of the excited electrons. Since different tissue structures have different molecular bonds, the Coblation® device may be configured to produce energy sufficient to break the molecular bonds of certain tissue but insufficient to break the molecular bonds of other tissue. For example, fatty tissue, (e.g., adipose) tissue has double bonds that require an energy level substantially higher than 4 eV to 5 eV (typically on the order of about 8 eV) to break. Accordingly, the Coblation® technology generally does not ablate or remove such fatty tissue; however, it may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of this phenomena can be found in commonly assigned U.S. Pat. Nos. 6,355,032, 6,149,120 and 6,296,136, the complete disclosures of which are incorporated herein by reference.

The active electrode(s) of a Coblation® device are preferably supported within or by an inorganic insulating support positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

In some embodiments, the active electrode(s) have an active portion or surface with surface geometries shaped to promote the electric field intensity and associated current density along the leading edges of the electrodes. Suitable surface geometries may be obtained by creating electrode shapes that include preferential sharp edges, or by creating asperities or other surface roughness on the active surface(s) of the electrodes. Electrode shapes according to the present invention can include the use of formed wire (e.g., by drawing round wire through a shaping die) to form electrodes with a variety of cross-sectional shapes, such as square, rectangular, L or V shaped, or the like. Electrode edges may also be created by removing a portion of the elongate metal electrode to reshape the cross-section. For example, material can be ground along the length of a round or hollow wire electrode to form D or C shaped wires, respectively, with edges facing in the cutting direction. Alternatively, material can be removed at closely spaced intervals along the electrode length to form transverse grooves, slots, threads or the like along the electrodes.

Additionally or alternatively, the active electrode surface(s) may be modified through chemical, electrochemical or abrasive methods to create a multiplicity of surface asperities on the electrode surface. These surface asperities will promote high electric field intensities between the active electrode surface(s) and the target tissue to facilitate ablation or cutting of the tissue. For example, surface asperities may be created by etching the active electrodes with etchants having a pH less than 7.0 or by using a high velocity stream of abrasive particles (e.g., grit blasting) to create asperities on the surface of an elongated electrode. A more detailed description of such electrode configurations can be found in U.S. Pat. No. 5,843,019, the complete disclosure of which is incorporated herein by reference.

The return electrode is typically spaced proximally from the active electrode(s) a suitable distance to avoid electrical shorting between the active and return electrodes in the presence of electrically conductive medium. In some embodiments described herein, the distal edge of the exposed surface of the return electrode is spaced about 0.5 mm to 25 mm from the proximal edge of the exposed surface of the active electrode(s), or about 1.0 mm to 5.0 mm. Of course, this distance may vary depending on the voltage ranges, conductive medium being used, and depending on the proximity of tissue structures to active and return electrodes. The return electrode will typically have an exposed length in the range of about 1 mm to 20 mm.

The current flow path between the active electrodes and the return electrode(s) may be generated by submerging the tissue'site in an electrical conducting medium (e.g., within a viscous medium, such as an electrically conductive gel) or by directing an electrically conductive medium along a medium path to the target site (i.e., a liquid, such as isotonic saline, hypotonic saline or a gas, such as argon). The conductive gel may also be delivered to the target site to achieve a slower more controlled delivery rate of conductive medium. In addition, the viscous nature of the gel may allow the surgeon to more easily contain the gel around the target site (e.g., rather than attempting to contain isotonic saline). A more complete description of an exemplary method of directing electrically conductive medium between the active and return electrodes is described in U.S. Pat. No. 5,697,281, previously incorporated herein by reference.

Alternatively, the body's natural conductive fluids, such as blood or extracellular saline, may be sufficient to establish a conductive path between the return electrode(s) and the active electrode(s), and to provide the conditions for establishing a vapor layer, as described above. However, conductive medium that is introduced into the patient is generally preferred over blood because blood will tend to coagulate at certain temperatures. In addition, the patient's blood may not have sufficient electrical conductivity to adequately form a plasma layer in some applications. Advantageously, a liquid electrically conductive medium (e.g., isotonic saline) may be used to concurrently “bathe” the target tissue surface to provide an additional means for removing any tissue, and to cool the region of the target tissue ablated in the previous moment.

The power supply, or generator, may include an interlock for interrupting power to the active electrode(s) when there is insufficient conductive medium around the active electrode(s). This ensures that the instrument will not be activated when conductive medium is not present, minimizing the tissue damage that may otherwise occur. A more complete description of such an interlock can be found in commonly assigned, U.S. Pat. No. 6,235,020, the complete disclosure of which is incorporated herein by reference.

The present invention may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In one configuration, each individual active electrode in the electrode array is electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form selected active electrodes (e.g., titanium or a resistive coating on the surface of metal, such as platinum).

It should be clearly understood that the invention is not limited to electrically isolated active electrodes, or even to a plurality of active electrodes. For example, the array of active electrodes may be connected to a single lead that extends through the catheter shaft to a power source of high frequency current.

The electrically conductive medium should have a threshold conductivity to provide a suitable conductive path between the return electrode and the active electrode(s.) The electrical conductivity of the medium (in units of millisiemens per centimeter or mS/cm) will usually be greater than 0.2 mS/cm, preferably will be greater than 2 mS/cm and more preferably greater than 10 mS/cm. In an exemplary embodiment, the electrically conductive medium may be isotonic saline, which has a conductivity of about 17 mS/cm. Applicant has found that a more conductive medium, or one with a higher ionic concentration, will usually provide a more aggressive ablation rate. For example, a saline solution with higher levels of sodium chloride than conventional saline (which is on the order of about 0.9% sodium chloride) e.g., on the order of greater than 1% or between about 3% and 20%, may be desirable. Alternatively, the invention may be used with different types of conductive media that increase the power of the plasma layer by, for example, increasing the quantity of ions in the plasma, or by providing ions that have higher energy levels than sodium ions. For example, the present invention may be used with elements other than sodium, such as potassium, magnesium, calcium and other metals near the left end of the periodic chart. In addition, other electronegative elements may be used in place of chlorine, such as fluorine.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck. The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation.) Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode). Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive medium.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with, e.g., lasers claiming small depths of necrosis, which are generally pulsed about 10 Hz to 20 Hz). In addition, the duty cycle (i.e., cumulative time in any one-second interval that energy is applied) is on the order of about 5.0% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes.

The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood).

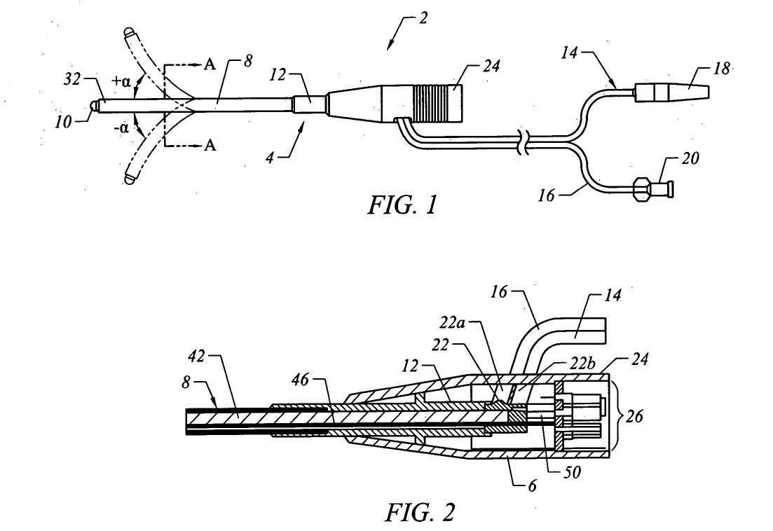

Referring now to the drawings, particular embodiments of the system and methods of the present invention are described. FIG. 1 illustrates a system 2 generally including a probe or wand 4 having a handle portion 6 at a proximal end and a shaft 8 extending distally from handle portion 6. A working tip 10 is provided at the distal end of shaft 8. Extending laterally from handle portion 6 is an aspiration tubing 14 and a conductive medium tubing 16. A connector 18, such as a universal connector, for coupling with a source of suction is provided at the proximal end of aspiration tubing 14 is. A connector 20, such as a Luer lock connector, for coupling to a source of conductive medium, such as saline, is provided at the proximal end of conductive medium tubing 16. Each of the connectors 18 and 20 may be provided with valve means to control the air pressure and fluid (or substance) flow therethrough, respectively.

Wand 4 is a representation of wands of the present invention, in this variation, the handle 6 is adapted to connect with a cable or line to a power supply or controller (not shown.) Alternatively, the handle 6 may be integrated with such a cable. The length, dimensions, and characteristics (e.g., the number of electrode, fluid delivery source, suction lumen) of the wand will depend upon-the particular application for which the wand is intended.

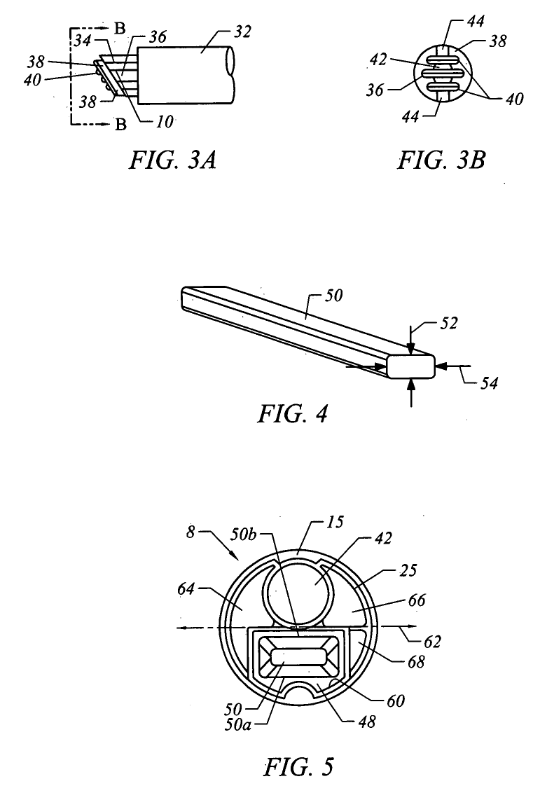

FIG. 2 illustrates a longitudinal cross-sectional view of the proximal portion of a variation of the invention. As illustrated, a support tube 12 may fixedly interconnect handle 6 and shaft 8. A manifold 22 may be positioned at the proximal end of support tube 12. Manifold 22 has an internal configuration and tubular extensions 22a and 22b which establish a fluid connection between aspiration tubing 14 and irrigation or conductive medium tubing 16, respectively, to corresponding aspiration and irrigation channels or lumens (see FIG. 3C) housed within support tube 12 and shaft 8. While the irrigation lumen is not shown in the view provided by FIG. 2, aspiration channel 42 is shown extending along the longitudinal axis of shaft 8 and tubular support 12 and terminates at an opening within or adjacent to the working tip 10.

In use, electrically conductive medium may be delivered through an irrigation lumen to the active and/or return electrodes. Alternatively, the medium may be present in the operative field or the device may be coated with an electrically conductive medium prior to activation. The aspiration lumen aspirates the excess conductive medium and/or tissue debris from the distal end of wand 4 or from the surgical site. In one embodiment, the fluid delivery and aspiration lumens create a recirculation system for minimizing the amount of conductive medium that contacts the patient, and for reducing the temperature to which a target tissue is exposed during a procedure.

As shown in FIG. 2, a connector 26 is seated within the proximal end 24 of handle 6. Connector 26 is adapted to interface with a power supply (e.g., a high frequency power supply) or controller via a cable. As mentioned above, the connector may be integral with a cable. Connector 26 provides the electrical contacts to the electrode leads or wires extending within and along support tubing 12 and shaft 8 to working tip 10.

An example of an active and return electrode configuration for use with the present invention is illustrated in FIGS. 3A and 3B. FIG. 3A illustrates a side view of the distal end 32 of shaft 8 having a distally extending working tip 10. In this embodiment, working tip 10 has a generally truncated cylindrical configuration having an angled tissue treatment surface 34 to optimize contact between the electrodes and the target tissue area; however, any other suitable geometry may be used for working tip 10. Positioned at the tissue treatment surface 34 are three active electrodes 40, as best shown in the end view of FIG. 3B in the direction of arrows B-B of FIG. 3A. While three active electrodes are illustrate, one or more may be employed. Electrodes 40 may be conductive members which extend through an electrically insulating electrode support member or spacer 38 which preferably comprises an inorganic support material (e.g., ceramic, glass, glass/ceramic, etc.). Spacer 38 separates active electrode terminals 40 from the return electrode 36. As illustrated, in some variations the return electrode is located on the instrument shaft such as circumferentially about support material 38.

In other variations, the return electrode may be placed proximal to the active electrodes but distal to the shaft. Other variations include electrode configurations where the return electrode is placed on the tissue treatment surface or even distal to the active electrodes. For example, see U.S. Provisional application No. 60/408,967, filed Sep. 5, 2002, entitled Method and Apparatus for Treating Vertebral Discs, the entirety of which is hereby incorporated by reference. Return electrode 36 and active electrodes 40 are coupled proximally through shaft 8 and support tube 12 terminating proximally with the connector 30.

It is noted that the electrode configuration illustrated in FIG. 3A is for exemplary purposes only. The inventive wand may incorporate a number of different electrode configurations as illustrated by various patents cited herein and incorporated by reference. Moreover, in a simple variation, the invention described herein may be provided as a single active electrode bipolar device or a monopolar device.

FIG. 2A illustrates one variations of a reinforcing member 50 for use with the present invention. In this variation the reinforcing member 50 is an orientation beam or rod or the like 50 that extends the length of wand 4 from the distal end of shaft 8 to connector board 26. While providing some rigidity to wand 4, beam 50 is made of a material such that it is substantially malleable. Suitable materials include a relatively malleable, pliable or deformable stainless steel, such as 304 stainless steel, a shape memory material, such as nitinol in its martinsitic form, or the like.

Another feature of this beam 50 is its cross-sectional configuration, as best illustrated in FIG. 4, which includes a minor axis or dimension (i.e., thickness) 52 and a major axis or dimension (i.e., thickness) 54, where the major axis 54 is greater than the minor axis 51. Beam 50 is substantially bendable only about the major axis 54 and, thus, is orientable only within a single plane. Preferably, the ratio of the major axis dimension to the minor axis dimension is selected depending upon the application of the wand. In any case, the ratio is selected to minimize bending of the beam 50 within a single plane while maintaining size constraints for the particular application of the wand. For example, a wand designed to reach smaller regions. Such a ratio facilitates bending of the shaft in the intended plane while resisting bending in all directions or planes other than the intended plane. Additionally, beam 50, and thus shaft 8, is bendable within an angle a ranging from about −90° to about +90°, and more typically from about −45° to about +45°, as illustrated in FIG. 1. Orientation markers may be employed on the outside surface of shaft 8 to indicate the plane in which shaft 8 may be bent. The user may also palpate or feel the shaft to tactilely determined the orientation of beam 50 so as to initiate bending along the proper axis.

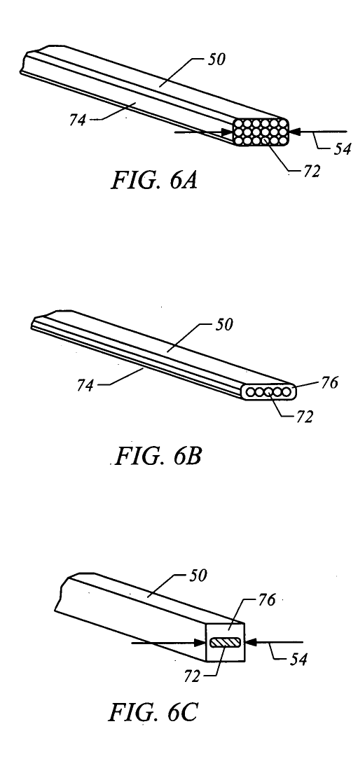

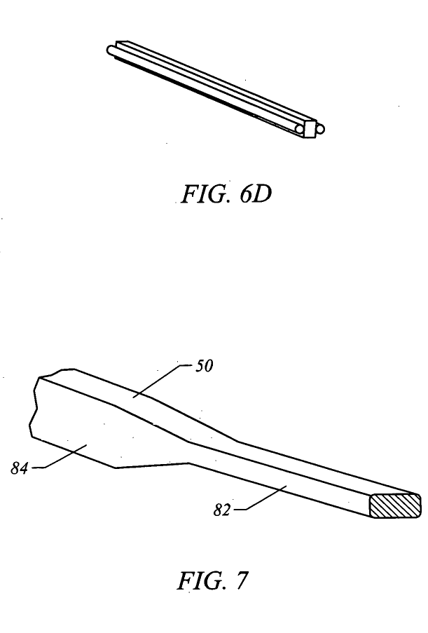

FIGS. 6A-6D illustrate end views of additional variations of reinforcing members of the present invention.

FIG. 6A illustrates a reinforcing members 50, composed of a plurality of support members 72 (such as beams or rods) which extend lengthwise through the reinforcing member 50. Optionally, the reinforcing members will have a covering 74 to join the support members 72 to function as essentially one structure. As illustrated, the minor axis 52 may be less than the major axis 54 to permit bending as described above.

FIG. 6B illustrates another variation of a reinforcing member 50 having one or more support members 72 extending lengthwise through the reinforcing member 50 which is also encased within a filler material 76 (e.g., a flexible epoxy) and surrounded by a covering 74. As noted in FIG. 6C, the cross section of the filler material 76 and covering 74 may be symmetrical about the major and minor axis, however, the variation may be adapted to have one or more support members 72 to facilitate bending as discussed herein (e.g., having more of a thickness along the major axis.)

FIG. 6D illustrates yet another variation of a reinforcing member 50 of the present invention. In this variation, the beam 50 comprises one or more center supports 78. The center reinforcing member 78 may be of a circular, square, rectangular, etc. cross section. The reinforcing member of this variation also includes one or more ribs 80 along the major axis 54 of the reinforcing member 50. The reinforcing member 50 of this variation may be extruded, welded, etc., to form the desired structure.

It is noted that the wand may be adapted to bend along the entire length of its shaft, or it may be adapted to bend only along a portion of its shaft, e.g., along a part of the distal end portion adjacent to the tissue treatment surface. In any case, the wand will be limited to bending within one plane. As illustrated in FIG. 7, the reinforcing member 50 comprises a distal portion 82 adapted to bend as described above, and a proximal portion 84 that is adapted to resist bending.

It is contemplated that the above described means that limit bending are not intended to be exhaustive rather exemplary. Additional variations of such reinforcing members may be incorporated into the inventive device. For example, the reinforcing member may be fabricated from a composite laminate material which bends substantially in one plane. It is also contemplated that an additional variation of the invention includes a shaft having a bending member integral thereto. Accordingly, the shaft may have a cross section or other portion that functions as a bending member and permits bending of the shaft in substantially one plane.

Due to the particular configuration and necessary orientation of the electrodes and the aspiration and conductive medium channels employed in wand 4, the arbitrary bending or orienting of the device may interfere with the proper functioning of the device, e.g., the electrodes may short, the fluid delivery and/or aspiration channels may become crimped, the fluid delivery may not deliver the conductive medium to the active electrodes, etc. Thus, limiting the movement of beam 50 to a single plane ensures proper functioning of system 2.

One possible arrangement of the various electrodes, lumens and orientation beam of the present invention is illustrated in FIG. 5 which provides a cross-sectional view of shaft 8 taken along arrows A-A of FIG. 1. Shaft 8 includes an outer sheath 15 and an inner sheath 25. Outer sheath 15 may be made of PVC, polyethylene, or a similar material. Inner sheath 25 includes a plurality of internal lumens running parallel to each other and having a particular arrangement wherein each lumen has a cross-sectional shape suitable for a designated function or purpose, e.g., to provide a fluid communication pathway for the transfer of gases and/or fluids or to provide a compartment for housing a hardware component, e.g., electrodes or beam, of the device. Inner sheath 25 is preferably made of an insulating material suitable for extrusion fabrication.

In certain embodiments, reinforcing member 50 is positioned within shaft 8 such that a surface 50a of reinforcing member 50 is substantially parallel to the reinforcing member's major axis is positioned against or adjacent an inner surface 60 of the shaft wall. The remaining components and lumens are laterally arranged relative to reinforcing member 50 to provide an arrangement or orientation such that their respective structures and/or functions are not impaired when reinforcing member 50 is operatively bent or oriented. For example, as illustrated in FIG. 5, the various lumens and components (i.e., electrodes) are laterally positioned counter clockwise from reinforcing member 50 as follows: lumen 64 defines the location of the active electrode, lumen 42 defines the aspiration channel through which suction is applied, lumen 66 defines the location of the irrigation channel and lumen 68 defines the location of the return electrode. While inner sheath 25 provides a defined arrangement of lumens and components, those skilled in the art will appreciate that more or fewer lumens and components may be provided in any arrangement wherein the proper functioning of the lumens and components are not compromised by the bending of reinforcing member 50. Additionally, the shape of the individual lumens may vary as necessary. For example, an inner lumen may have a notch to assist in assembly of the inner components of the wand.

It is noted that the present invention is useful in any kind of surgical application where restricting bending of an electrosurgical device is preferably limited to one plane. It is further noted that the present invention is particularly useful for treating tissue that is difficult to reach such as in the head and neck where the view and/or access of the target tissue area is completely or partially obstructed. Such difficult to view or reach target tissue areas may reside within the mouth, ear, pharynx, larynx, esophagus, nasal cavity and sinuses. Typical procedures involving these areas include tonsillectomies and adenoidectomies or other procedures which involve the removal of swollen or diseased tissue such as from the mucus linings, turbinates and/or neoplasms from the various anatomical sinuses of the skull, the epi-glottic and supra-glottic regions, and the salivary glands, as well as submucous resection of the nasal septum. Still yet, the present invention may also be useful for cosmetic and plastic surgery procedures in the head and neck, for, example, the ablation and sculpting of cartilage tissue, such as the cartilage within the nose that, is sculpted during rhinoplasty procedures.

An exemplary method of the present invention is now described in the context of a tonsillectomy and adrenoidectomy procedure, however, such example is not intended to be limiting to the invention as many applications and target tissues are treatable with the present invention. As will be apparent in the description below, a device according to the present invention that is deformable or bendable in substantially one plane is suitable for use in such applications.

After the patient is properly prepped and anesthetized, the surgeon assesses the direction or angle at which the target site, e.g., the tonsil to be removed (either partially or completely), is to be approached or accessed with the electrosurgical probe or wand 4. From such assessment, shaft 8 of wand 4 is bent or oriented at a selected location along the length of the shaft to achieve a selected angle. Such bending and orientation is accomplished while maintaining the proper configuration of the electrodes and patency of the irrigation and aspiration lumens of wand 4. The selected bending or orienting is accomplished by applying force to shaft 8 against a surface of reinforcing member 50 defining a major cross-sectional axis of reinforcing member 50 sufficient to bend reinforcing member 50 at a location proximal to the distal end portion to a desired orientation or angle. The proximal end of shaft 8 may be fixedly held to facilitate the selective bending or orientation.

The now angularly oriented shaft 8 is delivered through the access area and the active electrodes 40 are brought into contact with, or close proximity to, the target tonsil tissue. Typically, for accessing the tonsils, shaft 8 will be preferably be oriented downward at an angle from the axis defined by handle 6. The medical practitioner will bend the shaft 8 as desired, and within a single plane, depending upon the physiology and build of the patient. . Electrically conductive medium is then provided to the tissue treatment surface and electrodes. In the presence of electrically conductive medium a high frequency voltage is then applied between the active electrode terminals and the return electrode to generate a plasma field adjacent to the active electrodes, and to volumetrically remove or ablate at least a portion of the tonsil. The high frequency voltage generates electric fields around the active electrodes with sufficient energy to ionize the conductive medium adjacent to the active electrodes. Within the ionized gas or plasma, free electrons accelerate, and electron-atom collisions liberate more electrons. The process cascades until the plasma contains sufficient energy to break apart the tissue molecules, causing molecular dissociation and ablation of the tonsil tissue.

The surgeon may then repeatedly, as necessary, adjust the orientation of wand 4 as necessary to complete the intended ablation of the same tonsil, the second or may proceed to ablate the adenoids.

Kits

Also provided by the present invention are kits that include the electrosurgical devices as described above for use in a variety of surgical applications. The subject kits typically include instructions for using the subject systems in methods according to the subject invention. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the Internet, are provided. An example of this embodiment is a kit that includes a web address where the instructions can be viewed and/or from which the instructions can be downloaded. As with the instructions, this means for obtaining the instructions is recorded on a suitable substrate.

While the present invention has been described with reference to the specific embodiments thereof, it should be understood by those skilled in the art that various changes may be made and equivalents may be substituted without departing from the true spirit and scope of the invention. In-addition, many modifications may be made to adapt a particular situation, material, composition of matter, process, process step or steps, to the objective, spirit and scope of the present invention. All such modifications are intended to be within the scope of the present invention and the appended claims.

What is claimed is:

1. An electrosurgical probe for use with a power supply, comprising:
a shaft having an electrode assembly disposed on a distal end portion thereof comprising at least one active electrode;
at least one return electrode affixed to the probe
wherein the shaft includes a reinforcing member adapted to limit bending of the shaft to within substantially a single plane.

2. The electrosurgical probe of claim 1, wherein the reinforcing member is malleable.

3. The electrosurgical probe of claim 2, further comprising a tissue treatment surface located at the distal end portion, where at least a portion of the at least one active electrode is exposed at the tissue treatment surface, and where the return electrode is located proximal to the tissue treatment surface.

4. The electrosurgical probe of claim 2, wherein the return electrode is spaced from the at least one active electrode such that when the tissue treatment surface is brought adjacent a tissue structure immersed in electrically conductive fluid, the tissue treatment portion of the electrode terminal is positioned between the fluid contact surface of the return electrode and the tissue structure and the electrically conductive fluid completes a conduction path between the electrode terminal and the return electrode.

5. The electrosurgical probe of claim 1, wherein the at least one active electrode comprises a plurality of electrodes.

6. The electrosurgical probe of claim 1, wherein the reinforcing member comprises a malleable beam having a major cross-sectional dimension and a minor cross-sectional dimension, wherein the major cross-sectional dimension is sufficiently greater than the minor cross-sectional dimension so that less force is required to bend the shaft about the major cross-sectional dimension than is required to bend the shaft about the minor cross-sectional dimension.

7. The electrosurgical probe of claim 1, where the shaft is adapted to bend within substantially the single plane only along the distal end portion.

8. The electrosurgical probe of claim 9, where the reinforcing member has a distal cross sectional area greater than a proximal cross sectional area.

9. The electrosurgical probe of claim 1, wherein the shaft comprises a plurality of lumens arranged relative to the reinforcing member wherein the respective functions of the lumens are not significantly impaired when the reinforcing member is operatively bent or oriented.

10. The electrosurgical probe of claim 9, wherein at least one of the plurality of lumens comprises an aspiration lumen.

11. The electrosurgical probe of claim 9, wherein the plurality of lumens comprises an active electrode lumen and a return electrode lumen.

12. The electrosurgical probe of claim 9, wherein at least one of the plurality of lumens comprises a fluid delivery lumen having an opening towards the distal portion of the probe.

13. The electrosurgical probe of claim 12, where the fluid delivery lumen opening is adapted to deliver fluid between at least one of the active electrodes and the return electrode.

14. The electrosurgical probe of claim 12, further comprising a source of electrically conductive medium.

15. The electrosurgical probe of claim 1, where the reinforcing member includes at least one support member extending lengthwise through at least portion of the reinforcing member.

16. The electrosurgical probe of claim 15, where the reinforcing member further comprises a filler material.

17. The electrosurgical probe of claim 1, further comprising at least one rib along a side of the reinforcing member.

18. An electrosurgical probe, comprising:
a shaft having an electrode assembly disposed on a distal end portion thereof comprising at least one active electrode;
at least one return electrode affixed to the probe; and
a means for limiting bending of the shaft to within substantially a single plane.

19. The electrosurgical probe of claim 18, wherein said limiting means comprises a malleable reinforcing member.

20. The electrosurgical probe of claim 19, further comprising a tissue treatment surface located at the distal end portion, where at least a portion of the at least one active electrode is exposed at the tissue treatment surface, and where the return electrode is located proximal to the tissue treatment surface.

21. The electrosurgical probe of claim 19, wherein the shaft comprises a plurality of lumens arranged relative to the reinforcing member wherein the respective functions of the lumens are not significantly impaired when the reinforcing member is operatively bent or oriented.

22. The electrosurgical probe of claim 21, wherein at least one of the plurality of lumens comprises an aspiration lumen.

23. The electrosurgical probe of claim 22, wherein the plurality of lumens comprises an active electrode lumen and a return electrode lumen.

24. The electrosurgical probe of claim 21, wherein at least one of the plurality of lumens comprises a fluid delivery lumen having an opening towards the distal portion of the probe.

25. The electrosurgical probe of claim 24, where the fluid delivery lumen opening is adapted to deliver fluid between at-least one of the active electrodes and the return electrode.

26. The electrosurgical probe of claim 19, where the shaft is adapted to bend within substantially the single plane only along the distal end portion.

27. The electrosurgical probe of claim 18, wherein the at least one active electrode comprises a plurality of electrodes.

28. The electrosurgical probe of claim 18, wherein the shaft comprises a malleable reinforcing member having a major cross-sectional dimension and a minor cross-sectional dimension, wherein the major cross-sectional dimension is sufficiently greater than the minor cross-sectional dimension so that less force is required to bend the shaft about the major cross-sectional dimension than is required to bend the shaft about the minor cross-sectional dimension.
